(19) United States
(12) Patent Application Publication
(10) Pub. No.: US 2021/0008552 A1
(43) Pub. Date: Jan. 14, 2021

(54) NUCLEIC ACID EXTRACTION AND PURIFICATION CARTRIDGES

(71) Applicant: LEXAGENE, INC., Beverly, MA (US)

(72) Inventors: John REGAN, Boxford, MA (US); John DePiano, Burlington, MA (US)

(21) Appl. No.: 16/619,851

(22) PCT Filed: Jun. 14, 2018
(86) PCT No.: PCT/US2018/037620

§ 371 Date: Dec. 5, 2019

Related U.S. Application Data
(60) Provisional application No. 62/520,176, filed on Jun. 15, 2017.

(51) Int. Cl. B01L 3/00; G01N 1/40; C12Q 1/6806; G01N 1/10; G01N 35/10

(52) U.S. Cl. CPC B01L 3/502746; B01L 2200/10; B01L 3/567; C12Q 1/6806; G01N 1/10; G01N 35/1095; G01N 35/1009; B01L 2200/0631; B01L 2300/0681; B01L 2400/0644; B01L 2400/082; B01L 2400/0433; B01L 2300/123; B01L 2300/0663; G01N 1/405

(57) ABSTRACT

Nucleic acid extraction and purification cartridges and systems are provided. The cartridges can be removable and are configured to allow for the concentration of particles of interest, followed by nucleic acid extraction and purification. The cartridges directly contact samples and provide a partial barrier between samples and the reusable components of the system, thereby reducing the probability of clogging the system's microfluidics and fouling the lines, valves, and pumps of the system. Furthermore, these cartridges are designed to purity nucleic acids by removing the majority of inhibitors for down-stream genetic testing. Embodiments may comprise one, two, or three or more channels. In an exemplary embodiment the nucleic acid extraction and purification cartridge comprises a first channel containing a filter disposed therein; and a second channel containing a nucleic acid binding matrix disposed therein, wherein a first end of the cartridge is configured to directly contact a sample comprising a biological material, and wherein a second end of the cartridge Is configured to connect in a reversible fashion to a flow-through automated Instrument that controls fluid flow through the cartridge.

### BACKGROUND OF THE INVENTION

Some automated genetic analyzers are flow-through in nature, where liquid samples are drawn into the instrument's microfluidic channels via the action of pumps. These liquid samples are processed within the systems internal components and genetic analyses are performed before the resulting fluids are delivered to a waste receptacle. Some flow-through genetic analyzers utilize removable flow-through cartridges that are designed to concentrate particulate matter, including bacteria and viruses, before the genetic material is extracted and purified. These analyzers may be equipped with sensors to ensure proper placement and usage of the removable cartridges. Properly inserted flow-through cartridges create fluid- and air-tight seals with the flow lines of the analyzer. The flow-through nature of these removable cartridges allows for the processing of large-volume samples, which improves sensitivity for ultra-rare pathogen detection. Removable cartridges can use traditional filters, size exclusion filters, affinity filters, and the such, to capture the biological material of interest before the captured material is enzymatically treated and lysed and then passed over a nucleic acid binding matrix, where inhibitors are washed away. The DNA and RNA are then dried, and eluted from the matrix so they can be used in genetic tests.

Prior art flow-through removable cartridges are designed to clamp in-line with the system's microfluidics, such that there are lines (e.g., tubing) both before and after the cartridge. This design has two significant drawbacks. First, the design assumes the use of a reusable hollow needle, through which the sample is drawn into the instrument's microfluidics. This reusable component comes in direct contact with the sample, and must be decontaminated after every sample is processed such that the next sample does not experience carry-over contamination. This decontamination step significantly adds to the time required before the next sample can be processed. Such a slow-down reduces the throughput of the instrument, making it less valuable to the end-user. Also, decontaminating the outside of the hollow-needle requires either dispensing a volume of decontaminating fluid into the original sample container that is in excess of the original sample's volume or removing the original sample container and replacing it with a clean decontamination container that is narrower (i.e. less volume) and more easily filled with the decontaminating fluid. The former strategy requires the instrument to have access to very large volumes of decontamination fluids and the latter requires the end-user to manually replace the original sample container with a decontamination container before the decontamination step can be finished. This adds to the cost of the test, since either more decontamination fluid must be used or a separate decontamination container must be provided. If a separate decontamination container is provided, it makes it more likely that the end-user might forget this step, which increases the chances of incomplete decontamination, making the subsequent sample prone to carry-over contamination (and potentially a false positive result).

The second major drawback of prior art removable cartridges is that the permanent reusable components that the sample touches or flows through before reaching the cartridge (e.g., the hollow needle, instrument tubing, a valve, etc.) are prone to fouling and clogging since they are exposed to raw sample that potentially contains large particulate matter and fouling contaminants. Such a design is prone to frequent clogging, where samples fail to be successfully processed and instrument maintenance is required much more frequently. In addition, over time, fouling can reduce the performance of the instrument, which may lead to both false positive and false negative results.

To avoid these drawbacks, there is a need for a new cartridge design that increases the throughput of an instrument, reduces the requirement for extra steps by the end-user, reduces the volume of decontamination fluids that must be stored on the instrument (which also reduces cost), improves the robustness of the instrument, and reduces the chances of carry-over contamination (i.e. false positives). The invention described below meets these needs.

### BRIEF SUMMARY OF THE INVENTION

This invention removes the requirement to have a permanent hollow needle that is difficult to decontaminate by providing a removable nucleic acid extraction and purification cartridge that interfaces directly with the sample via a hollow line that is part of the disposable cartridge. The cartridges of the invention may comprise a filter inside the cartridge that may restrict the flow of large particulate matter and fouling contaminants into the more sensitive components of the re-usable portion of the instrument since these particulates and contaminants are largely retained within the cartridge. This design reduces the chances of clogging, reduces fouling, and makes it substantially easier to decontaminate the re-usable portions of the instrument Furthermore, there is no need to decontaminate a flow-through needle—since there isn't one—as ‘the needle portion’ is part of the removable cartridge and is simply disposed of at the conclusion of the sample being processed. As a result, decontamination fluids only need to be passed through the inside portions of the instrument that were exposed to the sample, but not the outside of any aspect (i.e. hollow needle) of the instrument. The cartridges of this invention can comprise one, two, three, or more channels. A single channel cartridge can be manufactured that completes all the necessary steps of sample concentration and nucleic acid extraction and purification. However, performance improvements can often be achieved by manufacturing cartridges that have two, three, or more channels, as the additional channels allows for the geographic separation of different functions within the cartridge, which can improve the efficiency of pathogen capture and nucleic acid extraction and purification.

In multi-channel cartridges, generally speaking, the first channel receives the sample first, followed by the second, and then the third channel—if present—and so on. Due to the possibility for a multiplicity of channels, the position of a functional component within the cartridge, say a nucleic acid binding matrix, may change from being located in the first channel to being located in the second channel or even the third channel for a one, two, and three channel cartridges, respectively.

In a first aspect the invention provides a nucleic acid extraction and purification cartridge comprising one channel. In some embodiments the cartridge comprises a channel containing a nucleic acid binding matrix disposed therein, wherein a first end of the cartridge is configured to directly contact a sample comprising a biological material, and wherein a second end of the cartridge is configured to connect in a reversible fashion to a flow-through automated instrument that controls fluid flow through the cartridge. In some embodiments the channel is functionalized with a ligand that binds to a target in a sample. In some embodiments the channel is configured to receive vibration and/or sonication to disrupt a sample. In some embodiments the channel contains particles disposed therein to aid in the disruption of a sample. In some embodiments the channel further contains a filter disposed therein. In some embodiments the filter comprises a porous material such as nylon, PTFE, or nitrocellulose. In some embodiments the filter is designed to retain targets of interest but allows other material, including fluids, to pass through. In some embodiments the nucleic acid binding matrix comprises silica, borosilicate glass, sol-gel, silanol-functionalized surface, or an anion-exchange resin. In some embodiments the nucleic acid binding matrix comprises a nucleic acid binding probe or aptamer. In some embodiments the cartridge is single-use.

In another aspect the invention provides a nucleic acid extraction and purification cartridge comprising two channels. In some embodiments the cartridge comprises a first channel containing a filter disposed therein; and a second channel containing a nucleic acid binding matrix disposed therein, wherein a first end of the cartridge is configured to directly contact a sample comprising a biological material, and wherein a second end of the cartridge is configured to connect in a reversible fashion to a flow-through automated instrument that controls fluid flow through the cartridge. In some embodiments the second channel is indirectly connected to the first channel through a sample container. In some embodiments the second channel is connected directly to the first channel. In some embodiments the second channel contains a dried reagent disposed therein for lysis of a sample. In some embodiments the dried reagent is an enzyme. In some embodiments the first channel is functionalized with a ligand that binds to a target in a sample. In some embodiments the first channel is configured to receive vibration and/or sonication to disrupt a sample. In some embodiments the first channel contains particles disposed therein to aid in the disruption of a sample. In some embodiments the filter comprises a porous material such as nylon, PTFE, or nitrocellulose In some embodiments the filter is designed to retain targets of interest but allows other material, including fluids, to pass through. In some embodiments the nucleic acid binding matrix comprises silica, borosiliate glass, sol-gel, silanol-functionalized surface, or an anion-exchange resin. In some embodiments the nucleic acid binding matrix comprises a nucleic acid binding probe or aptamer In some embodiments the cartridge is single-use.

In another aspect the invention provides a nucleic acid extraction and purification cartridge comprising at least three channels. In some embodiments the cartridge comprises a first channel that selectively enriches for targets of interest, a second channel containing a filter disposed therein; and a third channel containing a nucleic acid binding matrix disposed therein wherein a first end of the cartridge is configured to directly contact a sample comprising a biological material, and wherein a second end of the cartridge is configured to connect in a reversible fashion to a flow-through automated instrument that controls fluid flow through the cartridge In some embodiments the second and third channels are fluidically connected to the first channel. In some embodiments at least one of the channels contains a dried reagent disposed therein for lysis of a sample. In some embodiments the dried reagent is an enzyme. In some embodiments at least one of the channels is configured to receive vibration and/or sonication to disrupt a sample. In some embodiments at least one of the channels contains particles disposed therein to aid in the disruption of a sample. In some embodiments the filter comprises a porous material such as nylon, PTFE, or nitrocellulose. In some embodiments the filter is designed to retain targets of interest but allows other material, including fluids, to pass through. In some embodiments the nucleic acid binding matrix comprises silica, borosiliate glass, sol-gel, silanol-functionalized surface, or an anion-exchange resin. In some embodiments the nucleic acid binding matrix comprises a nucleic acid binding probe or aptamer. In some embodiments the cartridge is single-use. In some embodiments the first channel of the cartridge selectively enriches for targets of interest by having a surface functionalized with a ligand, such as an antibody or aptamer, or contains an I-shape pillar array, or is configured for spiral inertial microfluidics, acoustofluidic bacterial separation, deterministic lateral displacement (DLD), or other microfluidic techniques for enrichment.

In another aspect the invention provides an automated flow-through instrument comprising a controller, pumps, valves, flow channels, and a housing connector to reversibly connect a nucleic acid extraction and purification cartridge of the invention. In some embodiments the instrument is configured to detect a pressure change in a flow channel of the cartridge and to adjust a microfluidic protocol based on the pressure change. In some embodiments the instrument is configured to pass heated fluids and/or air through the cartridge. In some embodiments the instrument is configured to control vibration and/or sonication of the cartridge to promote sample disruption. In some embodiments the instrument contains a detector to detect a target enriched or bound to the ligand in the cartridge. In some embodiments the instrument contains a component for genetic amplification and detection of a target nucleic acid, including but not limited to qPCR, sequencing, digital PCR, and isothermal amplification. In some embodiments the instrument reports results. In some embodiments the instrument comprises containers to hold reagents and waste fluids. In some embodiments the instrument is configured to use a plurality of reagents selected from lysis buffer, wash buffer, alcohol, molecular amplification and detection reagents, enzymes, elution buffer, water, oil, decontamination fluid, and air. In some embodiments the instrument contains components to assist in the merging of two aqueous flow streams and the separation of an aqueous solution into segments that are kept separated in a flow channel using intervening air or oil. In some embodiments the instrument contains decontamination buffers to clean re-usable components that come in contact with sample after every sample is processed.

### BRIEF DESCRIPTION OF THE DRAWINGS

Items shown in figures

10—First Channel

11—Filter

11A—Support structure for filter (11)

12—Upper connector

12A—Line

12B—Connector

15—Particles for physical (mechanical) disruption

20—Second Channel

21−Nucleic acid binding matrix

22—Connector

30—Third Channel

31—Liquid sample

32—Sample container

40—Cartridge housing

41—Connector of cartridge to flow-through automated instrument

42—Connector of cartridge to flow-through automated instrument

43—Cartridge Housing Clamping Mechanism

44—Cartridge Housing Clamping Mechanism

50—Multi-position rotary valve

51—Port of multi-position rotary valve (50) connected to second channel of cartridge

52—Port of multi-position rotary valve (50) connected to first channel of cartridge

53—Port of multi-position rotary valve (50) connected to a line leading to lysis buffer reservoir

54—Port of multi-position rotary valve (50) connected to a line leading to alcohol reservoir

55—Pump line, which connects the central port (100) of rotary valve (50) to the pump (61)

56—Port of multi-position rotary valve (50) connected to a line leading to wash buffer reservoir

57—Port of multi-position rotary valve (50) connected to a line leading to elution buffer reservoir

58—Port of multi-position rotary valve (50) connected to a line leading to air

59—Port of multi-position rotary valve (50) connected to a line leading to bleach (decontamination fluid)

60—Port of multi-position rotary valve (50) connected to a line leading to other components of the microfluidic system

61—Pump

62—Port of multi-position rotary valve (50) connected to third channel of cartridge

70—Rotating rotor channel of the multi-position rotary valve (50)

71—Port of multi-position rotary valve (50) connected to a line leading to waste receptacle

81—Binding ligand, such as an antibody, aptamer, polysaccharide, etc. (may also be an I-shape pillar array, or component configured for spiral inertial microfluidics, acoustofluidic bacterial separation, deterministic lateral displacement (DLD), or other microfluidic techniques/component used to enrichment for targets of interest)

82—Detection component, such as surface plasmon resonance

100—Central port of the rotary valve (50)

111—Line connecting second channel of the cartridge to flow-through automated instrument

211—Line connecting first channel of the cartridge to flow-through automated instrument

101—Channel

101A—Feature along channel 101 for storing lyophilized enzyme(s)

201—Channel extension/lower line

301—Channel

301A—Feature along channel 301 for storing lyophilized enzyme(s)

500—Nucleic acid extraction and purification cartridge

501—Upper half of nucleic acid extraction and purification cartridge (500)

502—Gasket for nucleic acid extraction cartridge (500)

503—Lower half of nucleic acid extraction and purification cartridge (500)

FIG. 1A shows a schematic representation of an embodiment of a one channel nucleic acid extraction and purification cartridge.

FIG. 1B shows a schematic representation of an embodiment of a one channel nucleic acid extraction and purification cartridge.

FIG. 1C shows a cross-section of the one channel nucleic add extraction and purification cartridge shown in FIG. 1B.

FIG. 2A shows a schematic representation of an embodiment of a two-channel nucleic acid extraction and purification cartridge.

FIG. 2B shows a cross-section of the first channel of the nucleic acid extraction and purification cartridge shown in FIG. 2A.

FIG. 2C shows a cross-section of the second channel of the nucleic acid extraction and purification cartridge shown in FIG. 2A.

FIG. 2D shows a schematic representation of an embodiment of a two-channel nucleic acid extraction and purification cartridge.

FIG. 2E shows a schematic representation of an embodiment of a two-channel nucleic acid extraction and purification cartridge.

FIG. 2F shows a schematic representation of an embodiment of a two-channel nucleic acid extraction and purification cartridge.

FIG. 2G shows a cross-section of the first channel of the nucleic acid extraction and purification cartridge shown in FIG. 2F.

FIG. 2H shows a schematic representation of an embodiment of a two-channel nucleic acid extraction and purification cartridge.

FIG. 2I shows a vertically-separated view of an embodiment of a two-channel nucleic acid extraction and purification cartridge.

FIG. 2J shows a cross-section view of an embodiment of a two-channel nucleic acid extraction and purification cartridge.

FIG. 2K shows a side view of an embodiment of a two-channel nucleic acid extraction and purification cartridge.

FIG. 3A shows a schematic representation of an embodiment of a three-channel nucleic acid extraction and purification cartridge.

FIG. 3B shows a schematic representation of an embodiment of the first channel of the three channel nucleic acid extraction and purification cartridge shown in FIG. 3A.

### DETAILED DESCRIPTION OF THE INVENTION

### A. Nucleic Acid Extraction and Purification Cartridges

The nucleic acid extraction and purification cartridges of the invention comprise at least one channel. The ends of the channel allow for fluid communication at a first end with a sample and at a second end with a channel connected to the cartridge housing area of a flow-through automated instrument. The nucleic acid extraction and purification cartridges are configured to allow direct fluid communication with a sample. In some embodiments, the direct fluid communication with the sample is via an extension line connected to one end of the channel (for larger volume samples). In some embodiments, the channel connects the sample without use of a line (for lower volume samples). In some embodiments, the nucleic acid extraction and purification cartridge comprises no more than one channel. In some embodiments, the nucleic acid extraction and purification cartridge comprises two, three, or more channels.

Within the context of this invention, a “cartridge” is a disposable and removable unit that is incorporated into a system capable of automated flow-through microfluidic operations. Typically, the cartridge has one or more functions selected from concentrating samples, extracting and purifying the nucleic acids within a sample, and even determining whether an analyte, such as a toxin, bacteria, or virus, is present via binding to a ligand and detection by surface plasmon resonance or other type of detection component. Typically, a system contains one cartridge for every flow path of the instrument. For example, an instrument with 12 different flow paths is capable of processing twelve samples at a time. Accordingly, the instrument often has 12 separate cartridge housing areas, one for each flow path, although it is possible to envision a single cartridge that services all 12 flow paths, for example. A flow path is defined as the lines, valves, and pumps associated with processing a sample in the system. Nucleic acid extraction generally, but not always, requires the use of several lines within one flow path. These lines are often associated with a pump and a valve that directs fluid flow. These lines connect to the cartridge housing, which is specially designed to interface with an inserted cartridge. The lines entering the cartridge housing are effectively ‘open’ and the process of properly inserting and damping a cartridge into the housing chamber effectively connects these open lines with the cartridge ends, which are also ‘open’. In some embodiments these ‘open’ lines are effectively closed when a cartridge is not inserted, and they open when a cartridge is inserted. A properly inserted cartridge connects the channels of the cartridge with the permanent lines of the instrument to form flow paths. This connection allows for the instrument to pass fluid stored on the instrument (or air) through the cartridge to perform sample extraction and nucleic acid purification. Following purification, the nucleic acids are assembled into genetic reactions inside the instrument and the instrument processes these to completion and reports a result. Cartridge housings are generally placed in accessible locations, so cartridges may be easily and quickly exchanged if desired.

Within the context of this invention, a “channel” refers to defined flow path for fluids and/of air within a cartridge. A channel has at least two openings that connect its defined flow path to ends of the cartridge or to another channel within the cartridge. By way of non-limiting example, a cartridge may contain only a single channel and the ends of the channel may be located at opposite ends of the cartridge. In another non-limiting example, the cartridge may contain a second channel that intersects with the first channel. Fluids and/or air can be flowed through the channel in either direction under control of the pump(s) and/or valve(s) of a flow-through automated instrument.

In some embodiments, the cartridge channel is formed from different layers of substrate that are permanently or temporarily attached to each other, such as by bonding or clamping together.

In some embodiments the cartridge is fabricated in a planar substrate. Suitable substrate materials are generally selected based upon their compatibility with the conditions present in the particular operation to be performed by the cartridge. Such conditions can include extremes of pH, temperature, salt concentration detergents, and application of electrical fields. Additionally, substrate materials are also selected for their inertness to critical components of an analysis or synthesis to be carried out by the device.

Examples of useful substrate materials include, e.g., glass, quartz and silicon as well as polymeric substrates, e.g. plastics. In the case of conductive or semi-conductive substrates, it will generally be desirable to include an insulating layer on the substrate. This is particularly important where the device incorporates electrical elements, e.g., electrical fluid direction systems, sensors and the like. In the case of polymeric substrates, the substrate materials may be rigid, semi-rigid, or non-rigid, opaque, semi-opaque or transparent, depending upon the use for which they are intended. For example, devices which include an optical or visual detection element, will generally be fabricated, at least in part, from transparent materials to allow, or at least, facilitate that detection. Alternatively, transparent windows (e.g., glass or quartz) may be incorporated into the device for these types of detection elements. Additionally, the polymeric materials may have linear or branched backbones, and may be crosslinked or non-crosslinked. Examples of particularly preferred polymeric materials include: polydimethylsiloxanes (PDMS), polyurethane, polyvinylchloride (PVC) polystyrene, polysulfone, polycarbonate, polypropylene, and the like.

In some embodiments the length and the cross-section diameter of the channels are independently selected from 100 to 200 microns, 200 to 400 microns, 400 to 600 microns, 600 to 1,000 microns, and 1,000 to 2,000 microns. The cross-section of the channels may be any suitable shape, including without limitation, a circle, square or rectangle. Although sometimes shown as straight channels, it will be appreciated that in order to maximize the use of space on a substrate, or for other reasons, serpentine, saw tooth or other charnel geometries may be used, to incorporate effectively longer channels in shorter distances.

Manufacturing of the channels into the surface of the substrates may generally be carried out by any number of microfabrication techniques that are well known in the art. For example, lithographic techniques may be employed in fabricating, e.g., glass, quartz or silicon substrates, using methods well known in the semiconductor manufacturing industries such as photolithographic etching, plasma etching or wet chemical etching. Alternatively, micromachining methods such as laser drilling, micromilling, and the like may be employed. Similarly, for polymeric substrates, well known manufacturing techniques may also be used. These techniques include injection molding or stamp molding methods where large numbers of substrates may be produced using, e.g., rolling stamps to produce large sheets of microscale substrates or polymer microcasting techniques where the substrate is polymerized within a micromachined mold.

In some embodiments, the cartridges are made of a plastic material, such as polycarbonate, which is a relatively hard plastic that allows for more reproducible assembly of the cartridges. In contrast, the cartridge housing may be made of very hard plastic, such as polyether ether ketone (PEEK). When a cartridge is clamped in place, the cartridge's plastic end bends or molds to the shape of the PEEK plastic housing, thereby making a fluid and air-tight seal between the channels of the cartridge and the lines of the flow-through instrument. Also, a gasket may be used to improve the seal.

Within the context of this invention, a “filter” refers to a medium for separating solid from liquid. Typically, the liquid passes through the filter and the particulates (i.e. the pathogens and cells) in the liquid are retained on/in the filter, effectively concentrating the particulates in the liquid at a known location, where the microfluidic instrument can then direct lysis buffers to this zone to begin the DNA and RNA purification process. In some embodiments, the filter is a porous material comprised of nylon, nitrocellulose, PFTE, sol-gel, or other particle capturing material.

Within the context of this invention, a “sample” refers to a solution that contains (or is suspected of containing) nucleic acids. In some embodiments, the sample is blood, plasma, or other bodily fluids collected from humans or animals. In other embodiments, the sample is any solution that contains (or is suspected of containing) cells or portions of cells derived from samples taken from humans, animals, plants, fruits, vegetables, etc. that may have been homogenized or enzymatically separated. In some embodiments, the sample is water, liquid or fluid collected from beverage companies, ponds, lakes, streams, oceans, drinking water containers on farms, aquaculture pen water, and the like. In some embodiments, a swab is swirled inside a fluid container and the fluid is analyzed, as is common in food processing plants and food packaging plants.

Within the context of this invention, a “nucleic acid binding matrix” refers to a substrate capable of binding nucleic acids within a sample. Examples include silica, Whatman 1825-047 GF/F Borosilicate Glass Microfiber Filters, silanol-functionalized surfaces, silica sol-gel matrices, and anion-exchange resins.

Within the context of this invention, a “flow-through automated instrument” refers to a device comprised of a controller, microfluidic channels, tubing, pumps, valves, t-junctions, heated elements for assisting in biochemical reactions, optical elements for analyzing genetic reactions, and cartridge housing chambers for receiving cartridges of the invention, and the instrument has the ability to control the flow of fluids and/or air, using the pumps and valves, into and out of the microfluidic channels, tubing, and cartridges of the device to perform the desired tests.

Within the context of this invention, a “connector” refers to a position where two components of the system join. The connector may be a separate component or it may simply refer to a location where the two components join together. In some embodiments, the connector allows for a fluid and air-tight seal between two flow-through elements of the instrument/cartridge to be formed.

Within the context of this invention, a “housing chamber” refers to an area of a Row-through automated instrument into which a cartridge of the invention is inserted and clamped to create a fluid- and air-tight seal with the fluidic lines of the instrument.

Within the context of this invention, a “valve” refers to a device that regulates, directs or controls the flow of a fluid (gases, liquids) or gas (air) by opening, closing, or partially obstructing various passageways.

Within the context of this invention, a “fluidic system” refers to a connected series of passageways through which fluid may be passed. The fluidic system may include valves and connectors so that the passage of fluid is regulated and so that different components can be added or removed.

The nucleic acid extraction and purification cartridges comprise at least one channel comprising a nucleic acid binding matrix. The nucleic acid binding matrix is useful for the purification of nucleic acids from a sample.

The nucleic acid binding matrix may be highly structured to maximize the surface area for DNA- and RNA-binding, preferably in the range of 2.8 μg DNA/mg of matrix. The nucleic acid binding matrix may comprise or consist of any material that binds to nucleic acids. In some embodiments, the nucleic acid binding matrix binds to nucleic acids under certain conditions but not others, hence the binding is reversible. For example, in some embodiments, the nucleic acid binding matrix binds to nucleic acids in the presence of a high molarity chaotropic salt (such as guanidinium thiocyanate (GITC), guanidine thiocyanate (GuSCN), or guanidine hydrochloride (GuHCl)), but not in the presence of a low-salt aqueous solution such as water or a TRIS-buffer. In some embodiments, alcohol is mixed with the chaotropic salt to facilitate the binding of nucleic acids to the matrix. In some embodiments, the nucleic acid binding matrix comprises a silanol-functionalized surface, silica, borosilicate glass, or an anion-exchange resin. In some embodiments, the nucleic acid binding matrix comprises a surface functionalized with nucleic acid binding probes or aptamers. Nucleic acid binding aptamers may be chosen from any suitable molecule(s) known in the art. Examples include sequence-specific oligonucleotides and derivatives thereof. In some embodiments, the nucleic acid binding matrix is an anion-exchange resin, such as positively charged DEAE groups, cellulose, dextran, or agarose on the surface of the resin, where the salt and pH conditions determine whether DNA is bound or eluted.

In some embodiments, the nucleic acid extraction and purification cartridge comprises a plurality of channels, all of which may comprise a nucleic acid binding matrix, or alternatively, only a subset of the channels may comprise a nucleic acid binding matrix. Thus, for example, in an embodiment, the nucleic acid extraction and purification cartridge comprises one channel which comprises a nucleic acid binding matrix. In another embodiment, the nucleic acid extraction and purification cartridge comprises two channels, and one channel comprises a filter specifically designed to capture the particulates and the other channel comprises a nucleic acid binding matrix. In another embodiment, the nucleic acid extraction and purification cartridge comprises three channels, where one channel is used to concentrate particulates through an antibody-functionalized channel (or enrich targets through commonly used enrichment means), and the second channel is used to capture these particulates/targets on a filter, and the third channel comprises a nucleic acid binding matrix for DNA-RNA purification.

The nucleic acid extraction and purification cartridge comprises at least one channel comprising a nucleic acid binding matrix. In some embodiments, the nucleic acid binding matrix comprises a porous material made of silica, borosilicate glass, silica sol-gel, or some other nucleic acid binding surface, such as an anion-exchange resin.

In some embodiments the cartridge has only a single channel. In some embodiments the cartridge comprises a nucleic acid binding matrix and may also comprise a filter. In some embodiments, the filter impedes the flow of cells or cellular lysate. In some embodiments, the filter separates ceils, pathogens, proteins and/or lipids from aqueous solutions comprising target nucleic acids. In some embodiments, the filter is a 0.45 micron nylon filter. In some embodiments, the filter is a series of filters of progressively smaller pore size designed specifically to minimize the risk of complete clogging.

In some embodiments, the filter is functionalized by association with a probe(s), aptamer(s), ligand(s), or antibody(ies) that selectively binds to a target(s) and is used to enrich the target(s) from a sample. For example, an aptamer or antibody may bind to a particular cell type or pathogen present in a sample.

Typically, the cartridge is structured so that a first location on the surface of the cartridge contacts the sample (directly or indirectly) and second distant location on the surface of the cartridge connects, in a reversible fashion, to a flow-through automated instrument that controls fluid flow through the cartridge. The cartridge comprises a channel or channel network that fluidly connects a sample with a flow path of the flow-through automated instrument.

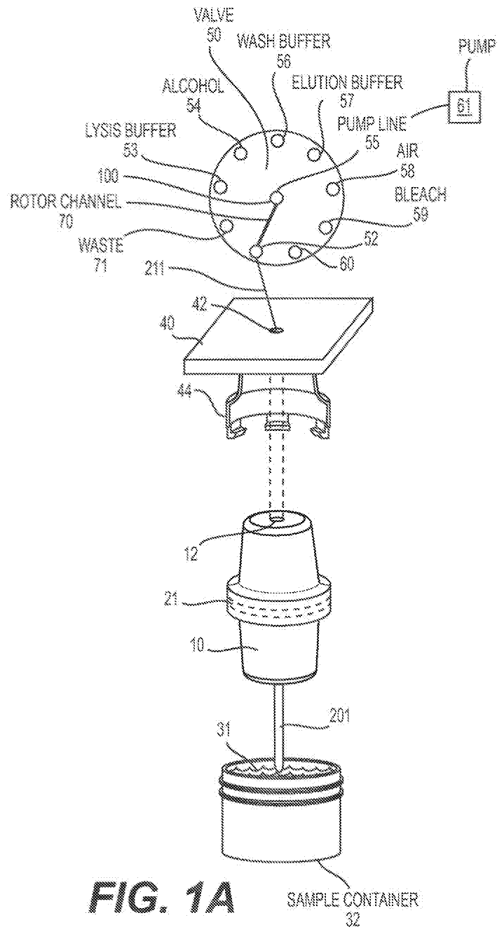

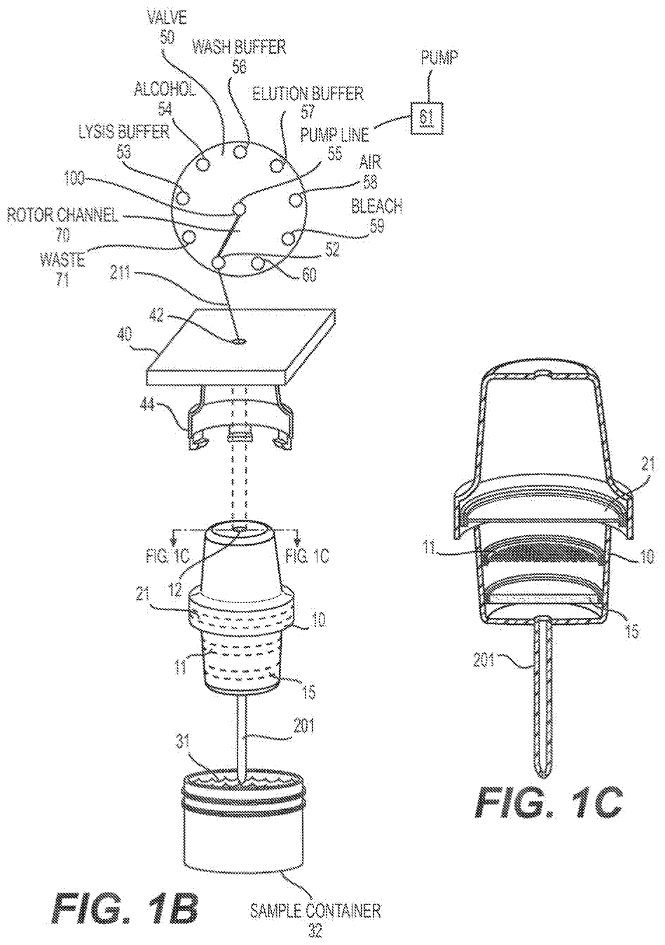

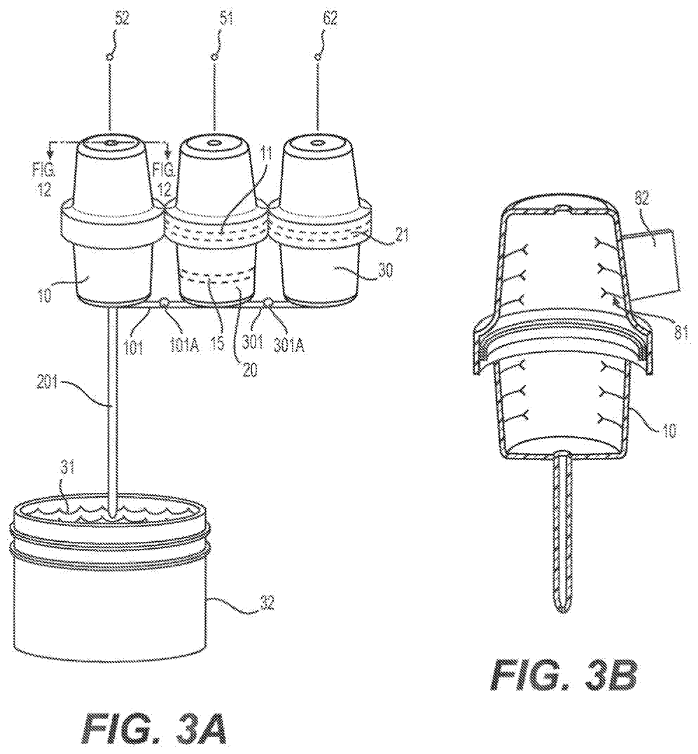

A first embodiment of the nucleic acid extraction and purification cartridge, is a single channel cartridge. The channel comprises a nucleic acid binding matrix, wherein a first end of the channel is configured to directly contact a sample comprising a biological material. Single channel embodiments are shown in FIGS. 1A to 1C. In some embodiments that are not shown in FIGS. 1A to 1C, the channel is functionalized with a ligand that binds to a target in a sample (as indicated in the first channel of the cartridge shown in FIG. 3A and in FIG. 3B) or the channel contains components/features to enrich the sample. In some embodiments, the channel is configured to receive vibration and/or sonication to disrupt a sample. In some embodiments the channel contains particles to aid in the disruption of a sample In some embodiments, the nucleic acid binding matrix is associated with a filter. In some embodiments, the filter comprises a porous material such as nylon, PTFE, or nitrocellulose. In some embodiments, the filer is designed to retain targets of interest but allows other material, including fluids to pass through. In some embodiments, the nucleic acid binding matrix comprises silica, borosilicate glass, a silanol-functionalized surface, or an anion-exchange resin. In some embodiments, the nucleic add binding matrix comprises a nucleic acid binding probe or aptamer. In some embodiments, an end of the cartridge connects in a reversible fashion to a flow-through automated instrument that controls fluid flow through the cartridge. In some embodiments, the cartridge is single-use and disposed of after a sample has been processed.

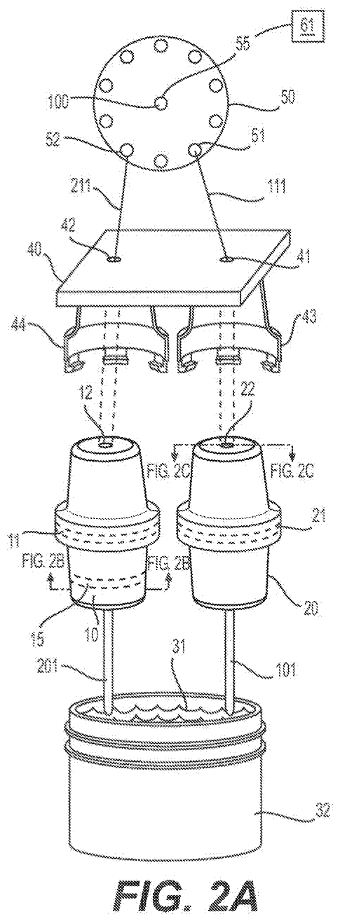

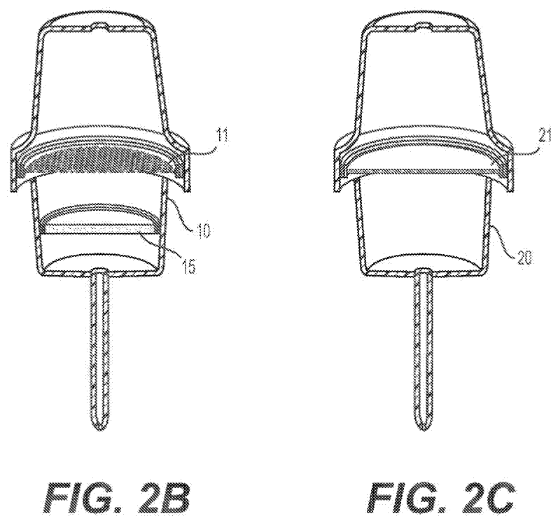

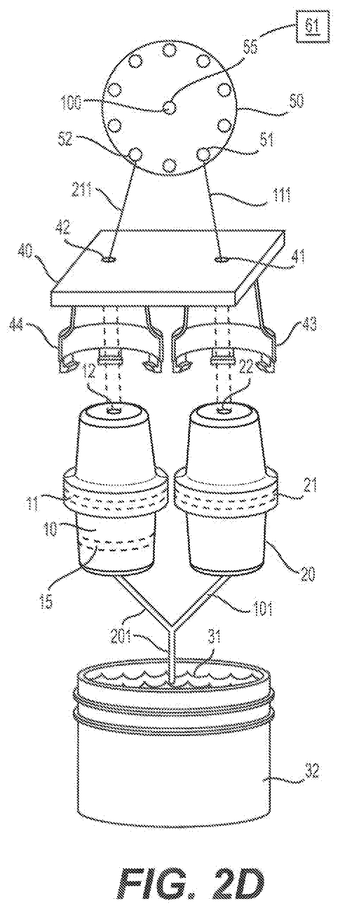

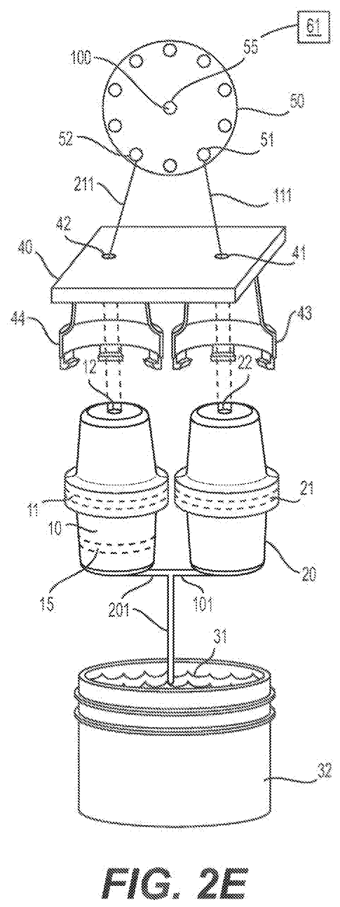

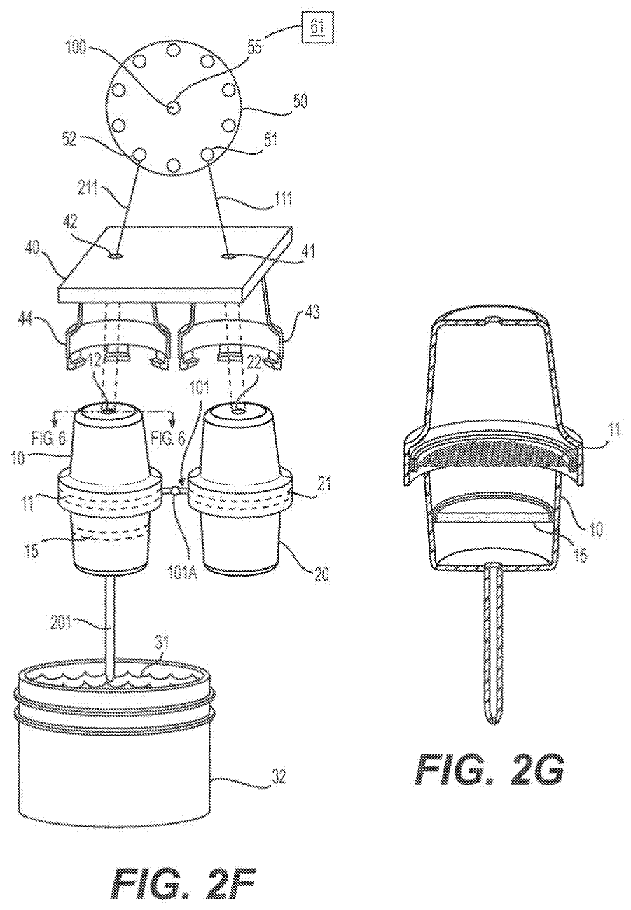

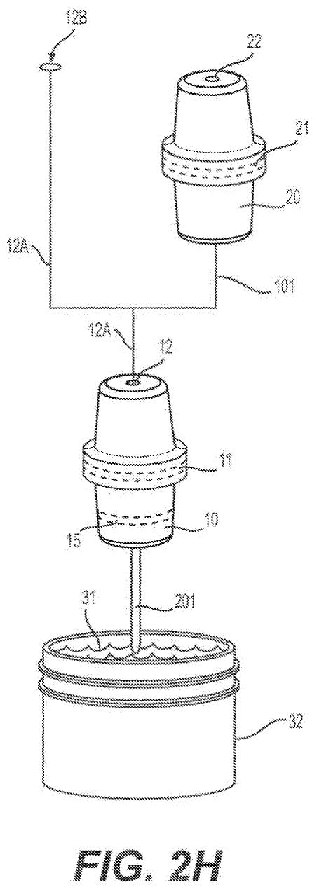

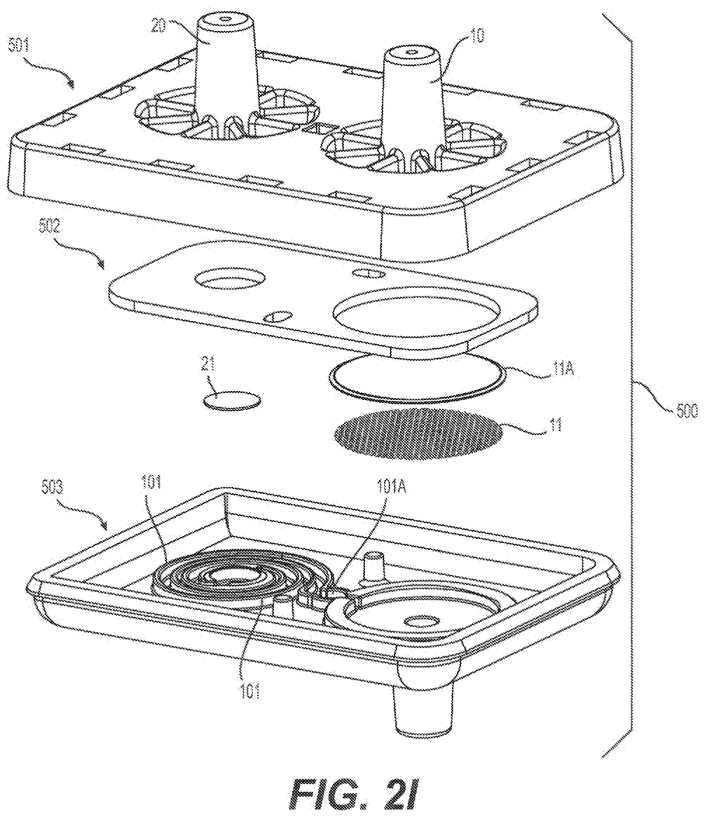

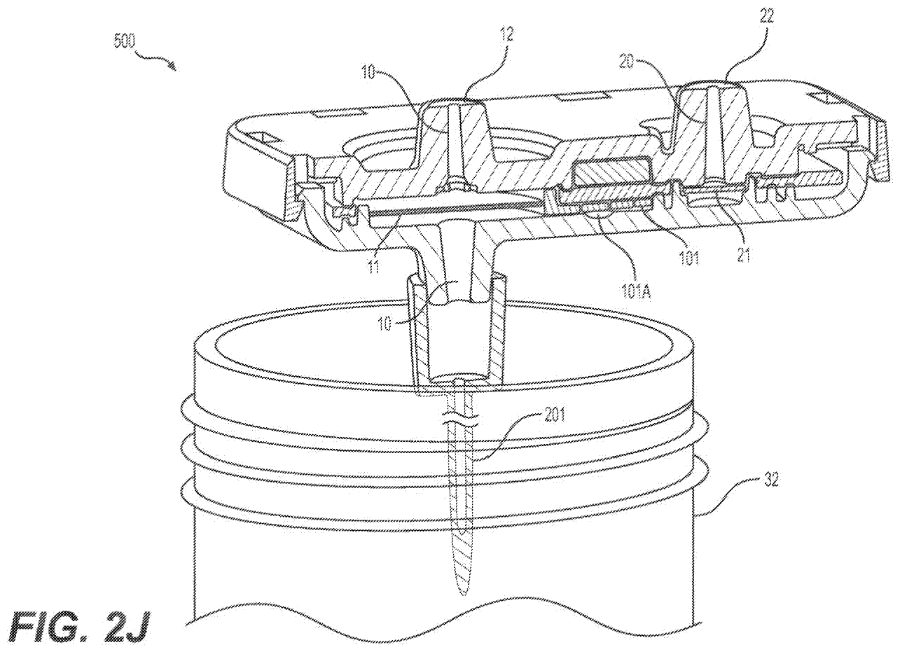

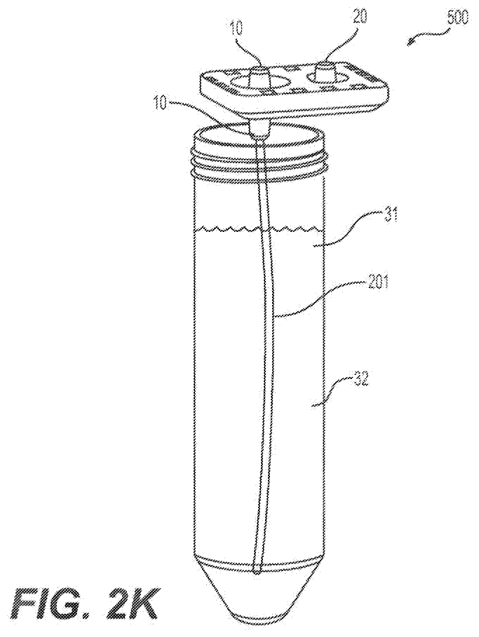

A second embodiment of the nucleic acid extraction and purification cartridge is a two-channel cartridge. For example, the nucleic acid extraction and purification cartridge may comprise a first channel comprising a filter, wherein a first end of the channel is configured to directly contact a sample comprising a biological material; and a second channel comprising a nucleic acid binding matrix. In some embodiments, the second channel is connected directly to the sample, in other embodiments the second channel is connected to the first channel. FIGS. 2A and 2C presents an embodiment in which one end of the second channel is configured to directly contact a sample comprising a biological material. In contrast, FIGS. 2D-2K present embodiments in which one end of the second channel is fluidically connected to the first channel. In some embodiments, the second channel contains a dried reagent for lysis of a sample. In some embodiments, the dried reagent for lysis of a sample is an enzyme.

A third embodiment of the nucleic acid extraction and purification is a three-channel cartridge. For example, the nucleic acid extraction and purification cartridge may comprise a first channel comprising a zone functionalized with ligands, wherein one end of the channel is configured to directly contact a sample comprising a biological material, a second channel comprising a filter; and a third channel comprising a nucleic acid binding matrix wherein the second and third channels are fluidically connected to the first channel. In some embodiments, the first channel is functionalized with a ligand that binds to a target in a sample. In some embodiments, the first channel is used to enrich the sample for targets of interest using established microfluidic techniques (e.g., spiral inertial microfluidic devices, acoustofluidic bacterial separation, I-shape pillar array, deterministic lateral displacement (DLD) technique, etc.). In some embodiments, the first channel is configured to receive vibration and/or sonication to disrupt a sample. In some embodiments, the first channel contains particles to aid in the disruption of a sample. In some embodiments, the first channel interfaces with a component on the system to allow for direct detection of bound ligands. In some embodiments, direction detection of bound ligands is either done optically or via a detection component such as surface plasmon resonance. In some embodiments, the second channel comprises a filter, which is comprised a porous material such as nylon, PTFE, or nitrocellulose. In some embodiments, the filter is designed to retain targets of interest but allows other material, including fluids, to pass through. In some embodiments, the second channel is connected directly to the first channel. In some embodiments, the third channel comprises a nucleic acid binding matrix that comprises silica, borosiliate glass, silanol-functionalized surface, or an anion-exchange resin. In some embodiments, the nucleic acid binding matrix comprises a nucleic acid binding probe or aptamer. In some embodiments, the second and/or third channel contains a dried reagent for lysis of a sample. In some embodiments, the dried reagent for lysis of a sample is an enzyme. In some embodiments, the third channel is connected directly to the second channel, and in other embodiments it is connected directly to the first channel. In some embodiments, the cartridge connects in a reversible fashion to a flow-through automated instrument that controls fluid flow through the cartridge. In some embodiments, the cartridge is single-use and disposed of after a sample has been processed.

In another aspect automated flow-through instruments for use with the cartridges of the invention are provided. In some embodiments the automated flow-through instruments are reversably connected to a nucleic acid extraction and purification cartridge of the invention to provide a flow-through nucleic acid extraction and purification system. In some embodiments the automated flow-through instrument comprises a controller, pumps, valves, flow channels, and a housing connector to reversibly connect the automated flow-through instrument to a nucleic acid extraction and purification cartridge of the invention. In some embodiments the instrument is configured to detect a pressure change in a flow channel of the cartridge and to adjust a microfluidic protocol based on the pressure change. In some embodiments the connected flow-through automated instrument is configured to pass heated fluids and/or air through the cartridge (and the air may be dehumidified). In some embodiments the connected flow-through automated instrument is configured to control vibration and/or sonication of the cartridge to promote sample disruption. In some embodiments the instrument contains a detector to detect a target bound to the ligand in the cartridge. In some embodiments the instrument contains a component for genetic amplification and detection of a target nucleic acid, including sequencing, qPCR, and digital PCR. In some embodiments the instrument reports results. In some embodiments the instrument comprises containers to hold reagents and waste fluids. In some embodiments the instrument is configured to use a plurality of reagents selected from lysis buffer, wash buffer, alcohol, molecular amplification and detection reagents, enzymes, elution buffer, water, oil, decontamination fluid, and air. In some embodiments the instrument contains components to assist in the merging of two aqueous flow streams and the separation of an aqueous solution into segments that are kept separated in a flow channel using intervening air or oil. In some embodiments the instrument contains decontamination buffers to clean re-usable components that come in contact with sample after every sample is processed.

FIG. 1A illustrates an embodiment of a nucleic acid extraction and purification cartridge that contains a channel (10) containing a nucleic acid binding matrix (21). The lower end of the channel is connected to a channel extension (201) that directly contacts liquid sample (31). The upper end the channel has a connector (12) that allows for connection of the cartridge to a connector (44) within the housing chamber (40) of a flow-through automated instrument. Within the instrument, line 211 connects the upper end of the channel to the fluidic system of the automated instrument such that it is in fluid contact with a valve (50). The valve (50) controls whether line 211 is in fluidic contact, through peripheral port (52), with the pump (61) of the automated instrument.

FIGS. 1B and 1C (a cross-section of 1B) illustrate a variation of FIG. 1A, where the channel (10) contains both a nucleic acid binding matrix (21) and a filter (11) for capturing particles of interest In such configuration, the spatial separation of the filter (11) and nucleic acid binding matrix (21) can be used to spatially separate the different functions (i.e. capture and lysis of the particulates versus purification of the nucleic acids), which can be advantageous. In some embodiments, there may also be a zone where particles (15) are held to aid in the disruption of the sample. In some embodiments, the channel may be functionalized with ligands to aid in the selective capture of a target(s) of interest (As indicated in FIG. 3B, but not shown in FIG. 1A-C or FIG. 2A-K). And the binding of this target may be detected by having the cartridge interface with a detector component of the flow-through instrument where bound particles are either detected in a direct optical manner or an indirect optical manner, such as plasmon surface resonance, or in a non-optical manner.

When the nucleic acid extraction and purification cartridge is reversibly connected to a microfluidic flow instrument, the instrument is used to control the flow of fluids through the cartridge. In the case of the single channel cartridges shown in FIGS. 1A and 1B, this is accomplished by applying suction or pressure to line 211, which is in fluid contact to the single channel.

In some embodiments, in addition to the nucleic acid binding matrix, the cartridge comprises a filter (11) that allows for selective capture of particular targets of interest, either small, medium, or large sized targets, using standard size exclusion techniques. In some embodiments, specific capture of targets of interest can be achieved by functionalizing the channel with a target specific ligand. In some embodiments, the cartridge is equipped with multiple filters or areas within the channel (10) that are designed to aid in sample disruption. For example, magnetic particles (15) that vibrate when applied to a rotating magnetic field generated by the instrument, could be added to help disrupt tough-to-break spores. Likewise, non-magnetic, but hard beads can be added and subjected to sonic blasts (sonication) to make them vibrate against one another, assisting in the lysis process. These physical features designed to aid in sample disruption can be positioned anywhere in the first channel, but most typically in the part of the channel closest to the sample. In alternative embodiments, they are integrated as part of the filter (11).

In these embodiments, the pump (61) is connected to the central port (100) of the multi-position rotary valve (50) via the pump line (55). The central port (100) connects to any one of the peripheral ports (52-54, 56-80, 71) via a valve rotor channel (70) that spins around the central port (100) like a hand of a clock. When the pump (61) applies suction or pressure, this force is transmitted through the pump line (55), through the rotor channel (70) of the multi-position rotary valve (50), and through the selected peripheral port (52-54, 56-60, 71), via the position of the rotor channel. To apply fluid flow into or out of the channel of the cartridge, the rotor channel (70), would be positioned at port 52, which is operatively connected to the channel (10).

FIGS. 2A-2C illustrate an embodiment of a nucleic acid extraction and purification cartridge that contains two channels (10) and (20). FIGS. 2B and 2C are cross sections of parts of FIG. 2A. The multi-position rotary valve connections are the same as in FIGS. 1A and 1B, but are not shown here to focus on the differences. The first channel (10) comprises a filter (11). The lower end of the first channel is connected to channel extension (201) that directly contacts a liquid sample (31). The upper end the first channel (10) has a connector (12) that allows for connection of the cartridge to a connector (44) within the housing chamber (40) of a flow-through automated instrument. Line 211 connects the upper end of channel 10 to port 52 of the fluidic system of the automated instrument such that it is in fluid contact with a valve (50) and the associated pump (61).

The second channel (20) comprises a nucleic acid binding matrix (21). The lower end of the second channel is connected to channel 101, which directly contacts the liquid sample (31). Because both line 201 and line 101 contact the sample, there can be fluid flow from 101 to 201 and vice versa, using the container (32) as a flow path connector. The upper end of the second channel has a connector (22) that allows for connection of the cartridge to a connector (43) within the housing chamber (40) of the flow-through automated instrument. Line 111 connects to the housing chamber (40) and connector (43) that allows the second channel (22) to come into contact with the fluidic system of the automated instrument including fluid contact with the valve (50) and the associated pump (61). The multi-position rotary valve (50) controls which port (51 or 52) is ‘open’ to allow for fluid flow through lines 111 and 211, respectively, of the automated fluidic instrument.

In comparison to the single channel configuration shown in FIGS. 1A, 1B, and 1C, the double-channel configuration of FIGS. 2A-2K allows for filtering of the sample at the filter (11) in a separate channel, which isolates the contaminates in a different channel than where the nucleic acid binding matrix is located, which makes the purification process easier and less prone to contamination.

FIGS. 2D-2E illustrate alternative embodiments of the cartridge shown in FIGS. 2A, 2B, and 2C. In FIGS. 2D-2E channel 10 contains a filter (11) and channel 20 contains a nucleic acid binding matrix (21) as in FIG. 2A. In FIGS. 2D and 2E channel 20 connects to channel 101 that in turn connects to channel 201. Channel 201 also connects to the sample and to channel 10. Thus, channels 10 and 20 are in fluidic contact via channels 101 and 201. Channels 10 and 20 are also in fluidic contact with the sample (31) via channels 101 and 201.

The embodiments illustrated in FIGS. 3 and 4 differ from each other only in the different geometry of the connections of channels 101 and 202. From a fluidic standpoint, they are nearly identical. Of course, several other geometries may be used.

Most features of the embodiment illustrated in FIG. 2F are the same as in the embodiments, illustrated in FIGS. 2D-2E, except FIG. 2F illustrates channels 10 and 20 in direct fluidic contact via channel 101. Alternative embodiments are possible. By way of example FIG. 2H shows an embodiment in which channel 101 connects from below the nucleic acid binding matrix (21) of the channel (20) to above the filter (11) of the channel (10). Alternatively, channel 101 may be positioned at the same level or above both the nucleic acid binding matrix and filter. In some embodiments the length of channel 101 is increased by utilizing an indirect pathway from channel 20 to channel 10 (see FIG. 2I). This increases the volume of channel 101 and allows eluting the nucleic acids off the nucleic acid binding matrix without having the eluate enter either of channels 10 and 201, which may contain sample inhibitors that could interfere with downstream genetic tests, as described below in Section C.

In addition, channel 101 may be designed to include certain features that permit the easy placement of lyophilized reagents, such as lysozyme (glycoside hydrolase) or proteinase K, which can be used to pre-treat retained particulates to help break down bacterial cell walls and spores. The feature might entail a widening of the flow-path, almost like a bubble, where the diameter of a lyophilized ball of enzyme(s) exceeds the diameter of the normal flow-path. Alternatively, filter or grid structures may keep the lyophilized component in place. Maintaining the lyophilized enzymes in a known location is important for reproducible solubilization of the enzymes. This feature could have been drawn in FIGS. 1A-1C, and 2A-2H, but has been omitted to focus only on certain features of each design.

FIG. 2H illustrates an alternative embodiment that is very similar to FIG. 2E, except that it shows the two channels spatially separated, rather than adjoined side-by-side. In this illustration the first channel (10) is shown below the second channel (20), but there is no reason these couldn't be reversed. This is shown to show various embodiments of the invention that might not at first appear obvious. In this case, a line (12A) extends from the top of the first channel up to a connector (12B), which would insert into the connector (44) for the cartridge housing (40), shown in FIG. 2A, for example.

FIG. 2I presents an exploded view of an embodiment of the nucleic acid extraction and purification cartridge (500). The cartridge is formed by joining layers 501, 502, and 503. Contrary to FIGS. 2A-2H, the orientation of this illustration is flipped. In this view, the first channel (10) is on the right and the second channel (20) is on the left. The configuration of flow paths and lines is similar to that shown in FIG. 2F. The filter comprises two layers (11 and 11A). Layer 11 is the filter designed to capture the particulate material, whereas layer 11A is support/backing designed to provide structural integrity behind (and/or above) 11. Such support provides structural stability to 11, which can be fragile. Also, shown in FIG. 2I is a feature that has a widen flow-path (101A) where lyophilized enzymes could be stored along the flow path of line (101). Also shown is how the line (101) does not need to be straight, but in fact can be curved to increase the volume of the flow path. In this case, it is shown wrapping around the feature that holds the nucleic acid binding matrix (21). The purpose of this longer flow path is to provide sufficient volume so that during nucleic acid elution, when the elution buffer is pushed back and forth over the nucleic acid binding matrix (21), the eluate never enters the area holding the filter (11) and its backing (11A), which might still contain remnants of lysis buffer, as this area is often not washed with the wash buffer. If the elution buffer picks up remnants of lysis buffer, the down-stream PCR reaction(s) could be inhibited.

In this illustration, a channel (101) contains a widen part (101A), where lyophilized enzyme may be stored. This reagent would be utilized in the following manner. After a sample has been passed over the filter (11) and the porous backing for the filter (11A) that provides structural support in the first channel (10), a low salt buffer, such as elution buffer (57), would be delivered down from the valve into the second channel (20), and pushed through channel (101) to reach the location of the lyophilized reagent (101A). The reagent would solubilize in the buffer, and the pump would continue to push the reagent into the first channel (10), where it would start to descend down channel (201). Before the solubilized enzymes reach the sample container (32), the pump would reverse directions, the valve would switch to port 52, and the solubilized enzyme would be drawn back toward the valve, where the enzymes can digest the cells and spores captured by the filter (11). This enzymatic treatment can greatly improve the efficiency of nucleic acid extraction The enzyme-treated sample would be drawn up into the valve (50) where it would be combined with a lysis buffer (53), before the lysis buffer and enzyme treated-sample is returned to the filter to further break apart particles of interest that are still stuck on the filter (11). Alternatively, the enzyme-treated sample could be parked in 101, while the lysis buffer is sent down channel 10, where it could be joined with the enzyme-treated sample that would be pushed out of 101 and into channel 10 to combine these two fluids.

FIG. 2J presents a cross-section view of an embodiment of the nucleic acid extraction and purification cartridge (503) shown in FIG. 2I. The configuration of flow paths and tines is the same as shown in FIG. 2I. The first channel (10) and second channel (20) are shown, as are the filter (11) and the nucleic acid affinity matrix (21). The channel extension/tower line (201) is shown as a separate piece of plastic that connects to the lower end of the first channel (10) and is dimensioned so that it can extend down into a sample container. Whether the lower line (201) and the channel (10) are two pieces of plastic that are fused or a single piece of plastic depends on the volume of the liquid sample (31) being processed and the ease of manufacturability.

FIG. 2K presents an alternative view of a nucleic acid extraction and purification cartridge (500) similar to that shown in FIG. 2J. In the embodiment represented in FIG. 2K, the channel extension/lower line (201) is longer and can extend to the base of a 50 ml conical tube (32) for large-volume sample processing. There is no reason this line can't be a meter or more in length depending on the sample volume being processed.

FIG. 3A illustrates a nucleic acid extraction and purification cartridge that contains three channels. In some embodiments, the first channel (10) is functionalized with ligands (81) to capture a target(s) of interest, the second channel (20) contains the filter (11), and the third channel (30) contains the nucleic acid binding matrix (21). In some embodiments, the first channel (10) on the far left is shown functionalized with antibodies (81) to capture targets of interest. In some embodiments, the first channel is configured to enrich the sample for targets of interest using established microfluidic techniques (e.g., spiral inertial microfluidic devices, acoustofluidic bacterial separation, I-shape pillar array deterministic lateral displacement (DLD) technique, etc.) The lower end of the first channel is connected to a line (201) that directly contacts the sample (31). In addition, line 101 operatively connects the second channel (20) with the first charnel (10), and line 301 operatively connects the third channel (30) with the second channel (20).

FIG. 3B illustrates the first channel (10) of the cartridge shown in FIG. 3A. In some embodiments, the first channel (10) comprises a channel functionalized with ligands (81) to bind targets of interest. The binding of targets to these ligands can be detected via a detection component (82) of the flow-through instrument. The detection component may provide direct visual detection or indirect detection, such as by surface plasmon resonance.

### B. Nucleic Acid Extraction and Purification Systems

The invention also provides nucleic acid extraction and purification systems comprising an automated flow-through instrument and a nucleic acid extraction and purification cartridge of the invention. In some embodiments the automated flow-through instruments are reversably connected to a nucleic acid extraction and purification cartridge of the invention to provide a system. In some embodiments the automated flow-through instrument comprises a controller, pumps, valves, flow channels, and a housing connector to reversibly connect the automated flow-through instrument to a nucleic acid extraction and purification cartridge of the invention. In some embodiments the instrument is configured to detect a pressure change in a flow channel of the cartridge and to adjust a microfluidic protocol based on the pressure change. In some embodiments the connected flow-through automated instrument is configured to pass heated fluids and/or air through the cartridge. In some embodiments the connected flow-through automated instrument is configured to control vibration and/or sonication of the cartridge to promote sample disruption. In some embodiments the instrument contains a detector to detect a target bound to the ligand in the cartridge. In some embodiments the instrument contains a component for genetic amplification and detection of a target nucleic acid, included by not limited to qPCR, sequencing, digital PCR, and isothermal amplification. In some embodiments the instrument reports results. In some embodiments the instrument comprises containers to hold reagents and waste fluids. In some embodiments the instrument is configured to use a plurality of reagents selected from lysis buffer, wash buffer, alcohol, molecular amplification and detection reagents, enzymes, elution buffer, water, oil, decontamination fluid, and air. In some embodiments the instrument contains components to assist in the merging of two aqueous flow streams and the separation of an aqueous solution into segments that are kept separated in a flow channel using intervening air or oil. In some embodiments the instrument contains decontamination buffers to clean re-usable components that come in contact with sample after every sample is processed.

### C. Use of the Nucleic Acid Extraction and Purification Cartridges and Systems

A skilled artisan will appreciate that the nucleic acid purification cartridges and systems of the invention may be substituted for prior art cartridges and systems in the numerous applications. The following applications are provided for illustration only and are not intended to be limiting.

In a first example, the nucleic acid purification cartridge shown in FIG. 1A is loaded into the cartridge housing (40) chamber of a flow-through microfluidic instrument by the operator such that the cartridge is clamped in-line with the system's microfluidics. The operator would also load a sample container (32) containing a liquid sample (31) to be analyzed. The operator would then interact with the system's graphical user interface to instruct to instrument to execute a series of microfluidic scripts to process the sample, including concentrating the sample over the nucleic acid binding matrix (21), and extracting and purifying the sample prior to joining the DNA/RNA with reagents for downstream genetic analysis.

The instrument processes the sample by coordinating the action of pumps and valves that direct fluid and air flow into the invention (the nucleic acid extraction and purification cartridge). Ultimately, the goal is to remove purified nucleic acids from the cartridge where they can be joined with other regents for downstream genetic analysis.

As way of example, a sample could be processed in the following manner using a one-channel cartridge, as shown in FIG. 1A. First, the pump (61) applies suction, assuming an airtight configuration, which allows suction to be applied directed through the pump line (55), the rotor channel (70), the upper line (211), the flow body (10), and line (201), which are ail connected, thereby applying a suction to the liquid sample (31), which gets pulled into the line (201) and drawn over the nucleic acid binding matrix (21). For this embodiment, the nucleic acid binding matrix serves as both a filter and nucleic acid binding surface. The particulate material in the sample (31) would be captured on the nucleic acid biding matrix (21). The efficiency of this would be determined by the pore size of the nucleic acid binding matrix (21). If desired, alt of the liquid sample (31) in the sample container (32) can be drawn over the nucleic acid binding matrix (21). If the volume of sample (31) exceeds the volume of the syringe connected to the pump (61), then the pump would need to go through multiple rounds of applying suction. After each round of applying suction, the fluid drawn through the cartridge and up into the valve (50) and into the pump line (55) can be re-directed to a waste receptacle (71), by changing the port of the multi-position rotary valve (50) to the waste port (71) and then pushing the fluid in the pump line (55) to the waste receptacle. If the sample (31) is large in volume, multiple rounds of drawing the sample in, then directing the ‘filtered’ sample to waste, would need to be completed until the sample container (32) is left empty.

Next, the multi-position rotary valve (50) changes location to port 53 and the pump (61) draws lysis buffer into the pump line (55), before the multi-position rotary valve returns to port 52 and pushes the lysis buffer down through the upper line (211) and over the nucleic acid binding matrix (21). The pump then pushes and pulls the lysis buffer back and forth over the nucleic acid binding matrix to dislodge and lyse the particulates of sample 31 that were retained by the filter.

Next, the instrument may be programed to then draw this lysate up into the valve where the rotary valve toggles back and forth between the upper line (211), where the lysate resides, and port 54, which is connected to an alcohol reservoir. Toggling back and forth allows for these two fluid streams to be interspersed in the pump line (55), where the simple flow of the fluids through the tubing results in complete mixing of these two liquids. Mixing is important, as combining alcohol to a high-salt lysate buffer is often required to provide the necessary chemical environment for nucleic acids to adsorb to the nucleic acid binding matrix. Next, the multi-position rotary valve (50) is directed to port 52, and the pump pushes the alcohol-mixed lysate through the upper line (211) and over the nucleic acid binding matrix (21), where the nucleic acids bind to the matrix. The alcohol-mixed lysed sample that has been stripped of its nucleic acids can then be pulled back up through the multi-position rotary valve and into the pump fine (55), before the valve repositions itself to the waste port (71) and the pump pushes this fluid to the waste receptacle. Of note, some embodiments of the invention will utilize more than one type of lysis buffer to achieve optimal lysis. Lysis buffers may include, but is not limited to, detergents, such as triton X100, NP-40, anionic surfactants, such as sodium dodecyl sulfate or sodium lauryl sulfate (SDS or SLS, respectively), urea, and/or chaotropic salts. It is not uncommon to use multiple lysis buffers. Doing so, would require additional ports on the multi-position valve to accommodate these additional fluids (not shown).

Next, the multi-position rotary valve (50) changes to port 56 and the pump draws an alcohol-containing wash buffer into the pump line (55), before the valve redirects to port 52 and the pump pushes the alcohol-containing wash buffer through the upper line (211) and the channel (10) containing the nucleic acid binding matrix (21). After washing the matrix to remove proteins, lipids, and inhibitors, the used wash buffer is pulled back up through the valve and into the pump line (55), before the valve repositions itself to the waste port (71) and the pump pushes this used wash fluid to the waste receptacle. Of note, some embodiments of the invention will utilize more than one type of wash buffer to achieve optimal washing. This would require additional ports on the multi-position valve to accommodate these additional fluids (not shown). Also not shown is the possibility for multiple waste receptacles such that incompatible fluids are kept separate.

Next, the nucleic acid binding matrix (21) is dried by positioning the valve to port 58, where the pump applies suction and pulls air into the pump line (55), before the rotor channel (70) is positioned to port 52, and the pump then pushes the air down the upper line (211) and over the nucleic acid binding matrix (21). Given only residual amounts of wash buffer is still associated with the nucleic acid binding matrix (21), as most of it was recovered and sent to waste (71), the residual wash (<10 μL) buffer can be expelled into the sample container (32). This process can be repeated until the nucleic acid binding matrix is sufficiently dry.

Next, the captured DNA and RNA on the nucleic acid binding matrix (21) is eluted, by positioning the valve to port 57, where the pump applies suction and pulls elution buffer into the pump line (55), before the rotor channel (70) is positioned to port 52, and the pump then pushes the elution buffer down the upper line (211) and over the nucleic acid binding matrix (21).

The eluted DNA and RNA is drawn up through the valve and into the pump line, before the valve redirects to port 60, which is connected to the rest of the microfluidic instrument so the DNA and RNA can be analyzed. Alternatively, the multi-position rotary valve (50) is equipped with additional ports (not shown) to permit the mixing of the eluted nucleic acids with other molecular reagents (e.g. master mix/super mix) at the valve. Then this mixture can be delivered out port 60 to meet up with other reagents (e.g. real-time PCR reagents) at a T-junction or other structure (e.g. cross-junction) before they are delivered to another part of the instrument for amplification and analysis Of course, other types of genetic analysis are also possible (e.g. sequencing, capillary electrophoresis, digital PCR, isothermal amplification, conventional PCR amplification, etc.)

Of note, the above description is only a general guide of how to purify nucleic acids from a liquid sample. Variations from the described protocol are likely required for optimal purification efficiency, but were omitted here for clarity via simplicity. For example, the multi-position rotary valve might have more ports than shown here. Often, it is desirable to treat the retained particulate matter with enzymes such as lysozyme and proteinase K before mixing the sample with a chemical lysis buffer. Also, it is often desirable to have more than one lysis buffer. Likewise, it is often desirable to have more than one wash buffer. Similarly, aspects of the instrument are not shown that may be used to improve the efficiency of extraction and purification. For example: 1) elements involved in heating the sample during/prior to lysis, 2) elements involved in sonicating or shearing the sample during lysis, 3) elements involved in sending heated or de-humidified air over the nucleic acid binding matrix that improve drying, and similar features are not shown, but can be assumed to be included to improve the overall performance of the instrument.

An advantage of the sample preparation cartridge shown in FIG. 1a is that it is extremely easy to manufacture and as such, it is low cost. It also occupies just one of the ports of the multi-position rotary valve, leaving the other ports available for the reagents required during sample extraction and purification. A disadvantage is that particulate capture, extraction, and purification happen on the same surface. Because of this, the nucleic acid binding matrix may not perform as well as desired for particulate capture. And in doing particulate capture, it might lower the efficiency of nucleic acid binding. For more optimal performance, it is generally advisable to have a specially designed filter for capturing the particulate matter in sample 31, and filtering and lysing the sample occurs prior to sending the lysate over the nucleic acid binging matrix.

For FIG. 1B, the sample would be processed in a very similar way, with one notable exception. In this embodiment, the particles are captured on a filter below the nucleic acid binding matrix. This confines the particulates to this area and minimizes the number of particulates that get through and bind to the nucleic acid binding matrix, which might reduce the binding efficiency of the nucleic acid binding matrix. In this embodiment, during the elution step, it would be beneficial to only pass the elution buffer over the nucleic acid binding matrix and stopping the flow before it passes over the lysed particulates captured on the filter as residual contaminants/impurities might still be retained on this filter and the elution buffer might release them into the purified nucleic acids, potentially causing trouble during down-stream genetic tests.

As way of example, a sample could be processed in the following manner using a two-channel cartridge as shown in FIG. 2, specifically looking at FIG. 2F (or FIGS. 2I, 2J, and 2K, which are effectively the same). First, the pump applies suction through the valve, the first upper line (211), the first channel (10), and the lower line (201) to draw the sample (31) over the filter (11). If the embodiment is equipped with a zone containing particles (15), which may or may not be magnetic, these particles would be vibrating to assist m disrupting the sample prior to reaching the filter (11). For this configuration after the particulates are captured on the filter (11), a lysis buffer is sent over the filter. In contrast to FIGS. 1A and 1B, where there was just one line accessing the filter, in this configuration, there are two lines accessing the filter, which permits two ways in which the lysis buffer can be delivered. First, the lysis buffer can be delivered in a fashion similar to FIGS. 1A and 1B, where the buffer is delivered down through the first upper line (211) aid first channel (10). Alternatively, the lysis buffer can be delivered through port 51, the second upper flow-through line (111), second channel (20), and line (101), and line 201 (and possibly even pooling in the original sample collection vessel (32)—assuming it has been emptied of sample), before the valve rotor position is changed to access port 52, and the pump is reversed. This action brings the lysis buffer up through the lower line (201) and first channel (10) to access the filter (11). This latter approach allows for the same direction of fluid flow as was required to originally pass the liquid sample (31) over the filter (11). The generated lysate containing the extracted nucleic acids can then be mixed with alcohol at the sample valve (50) before the mixture is delivered down line 111 to the nucleic acid binding matrix (21) within the second channel (20). Alternatively, the mixing of alcohol with the lysate could happen at the junction where 101 meets the first channel (10), where the alcohol is delivered down through line 111 and positioned in line 101. The pump would alternate pushing the lysate down the first channel (10) with pushing the alcohol out line 101 toward the first channel (10) and line 201 to have these two fluids mix within line 201 and possibly in the original sample vessel. This approach avoids bringing the lysate up into the valve, which could hasten the fouling of line 211, the valve rotor, and the pump line (55). This mixture would then be drawn up through 101 to bring the alcohol mixed lysate over the nucleic acid binding matrix (21).

Another option for this embodiment it to take advantage of feature 101A, which is designed to hold lyophilized enzymes to assist in lysing the sample. These enzymes would be accessed after the sample has been drawn over the filter (11), where the particles of interest are retained and prior to chemically lysing the sample with a lysis buffer. To access the lyophilized enzymes the pump (61) would draw elution buffer (FIG. 1A, port 57) into the pump line (55), then the valve would change to port 51 and the pump would push the elution buffer down through the second channel to the point of the lyophilized enzymes. In this case, the elution buffer is used to solubilize the lyophilized enzymes that are in feature 101A along line 101, but a buffer specifically for this purpose could also be used (requiring another port on the multi-position valve, which is not shown). The solubilized enzymes would then be pushed into the first channel (10), making their way down toward the sample container (32) via line 201. Before reaching the sample container, the multi-position rotary valve would turn to port 52 and the pump would apply suction, pulling the solubilized enzymes over the sample particulates retained on the filter (11). Here they would incubate (possibly at a specified temperature) to help break down cell walls and expose the inner nucleic acids. After the incubation, the lysis buffer would be mixed with the enzyme treated sample, in a fashion similar to the way alcohol is mixed with the lysis buffer. This could happen inside the valve or at the junction between line 101 and the first channel (10). To maximize performance, the enzyme-sample-lysate mixture would then be sent back over the filter (11) to ensure as much of the sample particulates are broken down and included in the lysate as possible before the lysate is mixed with the alcohol and sent over the nucleic acid binding matrix where the nucleic acids are bound.

Advantages of this configuration are that the filtration and sample disruption are confined to one channel and nucleic acid binding and purification are confined to a separate channel. This design allows for more efficient processing, since both the filter and nucleic acid binding matrix can be designed to maximize the performance of the two functions.

As way of example, a sample could be processed in the following manner using a three-channel cartridge, as shown in FIG. 3A. The pump would draw the sample up and through the first channel (10), where the ligands would capture targets of interest. The clarified sample held in the pump line (55) would be sent to waste (71). This process would be repeated until the sample vial is empty. If the first channel is equipped with a detection component (82), as shown in FIG. 3B, such as for surface plasmon resonance, the binding of targets to ligands would be detected. Possibly the system would be configured to continuously flow sample through the field of ligands in the first channel (10) . . . and only process the sample using the second channel (20) and third channel (30) if target has been confirmed to have bound to the ligand in the first channel (10). To process the captured targets, they need to be liberated from the ligands. This can be achieved by either flowing a protease through the channel or a lysis buffer. For the former, a lyophilized protease held in feature 101A could be solubilized by sending a buffer down through the second channel (20) and into line 201, before changing the active port on the multiposition valve to 52 and reversing the pump direction to bring the protease into the first channel (10) to liberate the target-ligand complexes from the solid support structure. These liberated targets would be then pushed into line 201, before changing the valve location to port 51 and drawing these targets over the filter (11) in the second channel. Alternatively, a protease could be stored in a reservoir accessible from the multiposition valve. For the latter, the pump draws lysis buffer into the pump line and delivers it down through the first channel (10). The lysed sample in line 201 could be retrieved through line 101, bringing the lysed targets of interest over the filter (11) in the second channel (20), where the particulate matter is concentrated. Now that the particles of interest are retained on the filter (11), processing of the sample for extraction and purification can proceed as previously described from there.

Possibly additional enzymes, such as a hydrolase or lysosome are stored in feature 301A, which can further customize the sample preparation process. Such enzymes could be solubilized as previously described and delivered over the filter. The remaining steps of combining an alcohol with the lysate and delivering this mixture to the nucleic acid binding matrix (21) contained in the third channel (30), would be completed in a similar fashion to what has already been described.

The purpose of showing this embodiment is to emphasize that it might be advantageous to have three channels for samples that might easily clog the filter (e.g. blood or particulate laden solutions) or for rapid protein-based detection using surface plasmon resonance. For complex matrices such as blood, it may be advantageous to configure the first channel with the necessary features to separate blood components to focus on one component (or pathogen in the blood). This might even involve actuating electrical impulses and different flow rates through features within the channel to achieve separation of the components. Published art has shown removal of blood cells, and concentration of pathogens into a flow stream using established microfluidic techniques (spiral inertial microfluidic devices, acoustofluidic bacterial separation, I-shape pillar array, deterministic lateral displacement (DLD) technique, etc.). This embodiment allows for selective capture of targets of interest, allowing the larger particulate matter to flow through the channel—ultimately going to the waste receptacle (71). Afterwards, the smaller targets of interest can be processed in the manner deemed most appropriate for the sample type and target of interest.

The foregoing embodiments, and the drawings are intended only as examples. No particular embodiment, drawing, or element of a particular embodiment or drawing is to be construed as a critical, required, or essential element or feature of any of the claims. Various alterations, modifications, substitutions, and other variations can be made to the disclosed embodiments, without departing from the scope of the present application, which is defined by the appended claims. The specification, including the figures, is to be regarded in an illustrative manner, rather than a restrictive one, and all such modifications and substitutions are intended to be included within the scope of the application. Steps recited in any of the method or process claims may be executed in any feasible order and are not limited to an order presented in any of the embodiments, or the claims.

1. A nucleic acid extraction and purification cartridge comprising:
a channel containing a nucleic acid binding matrix disposed therein,
wherein a first end of the cartridge is configured to directly contact a sample comprising a biological material, and
wherein a second end of the cartridge is configured to connect in a reversible fashion to a flow-through automated instrument that controls fluid flow through the cartridge.

2. The nucleic acid extraction and purification cartridge of claim 1, wherein the channel is functionalized with a ligand that binds to a target in a sample or features/elements that allow for specific target enrichment.

3. The nucleic acid extraction and purification cartridge of claim 1, wherein the channel is configured to receive vibration and/or sonication to disrupt a sample.

4. The nucleic acid extraction and purification cartridge of claim 1, wherein the channel contains particles disposed therein to aid in the disruption of a sample.

5. The nucleic acid extraction and purification cartridge of claim 1, wherein the channel further contains a filter disposed therein.

6. The nucleic acid extraction and purification cartridge of claim 5, wherein the filter comprises a porous material such as nylon, PTFE, or nitrocellulose.

7. The nucleic acid extraction and purification cartridge of claim 5, wherein the filter is designed to retain targets of interest but allows other material, including fluids, to pass through.

8. The nucleic acid extraction and purification cartridge of claim 1, wherein the nucleic acid binding matrix comprises silica, borosilicate glass, sol-gel, silanol-functionalized surface, or an anion-exchange resin.

9. The nucleic add extraction and purification cartridge of claim 1, wherein the nucleic acid binding matrix comprises a nucleic acid binding probe or aptamer.

10. The nucleic add extraction and purification cartridge of claim 1, wherein the cartridge is single-use.

11. A nucleic acid extraction and purification cartridge comprising:
a first channel containing a filter disposed therein; and
a second channel containing a nucleic acid binding matrix disposed therein,
wherein a first end of the cartridge is configured to directly contact a sample comprising a biological material, and
wherein a second end of the cartridge is configured to connect in a reversible fashion to a flow-through automated instrument that controls fluid flow through the cartridge.

12. The nucleic acid extraction and purification cartridge of claim 11, wherein the second channel is indirectly connected to the first channel through a sample container.

13. The nucleic acid extraction and purification cartridge of claim 11, wherein the second channel is connected directly to the first channel.

14. The nucleic acid extraction and purification cartridge of claim 11, wherein the second channel contains a dried reagent disposed therein for lysis of a sample.

15. The nucleic acid extraction and purification cartridge of claim 14, wherein the dried reagent is an enzyme.

16. The nucleic acid extraction and purification cartridge of claim 11, wherein the first channel is functionalized with a ligand that binds to a target in a sample or features/elements that allow for specific target enrichment.

17. The nucleic acid extraction and purification cartridge of claim 11, wherein the first channel is configured to receive vibration and/or sonication to disrupt a sample.

18. The nucleic acid extraction and purification cartridge of claim 11, wherein the first channel contains particles disposed therein to aid in the disruption of a sample.

19. The nucleic acid extraction and purification cartridge of claim 11, wherein the filter comprises a porous material such as nylon, PTFE, or nitrocellulose.

20. The nucleic acid extraction and purification cartridge of claim 11, wherein the filter is designed to retain targets of interest but allows other material, including fluids, to pass through.

21. The nucleic acid extraction and purification cartridge of claim 11, wherein the nucleic acid binding matrix comprises silica, borosiliate glass sol-gel, a silanol-functionalized surface, or an anion-exchange resin.

22. The nucleic acid extraction and purification cartridge of claim 11, wherein the nucleic acid binding matrix comprises a nucleic acid binding probe or aptamer.

23. The nucleic acid extraction and purification cartridge of claim 11, wherein the cartridge is single-use.

24. A nucleic acid extraction and purification cartridge comprising:
a first channel that selectively enriches for targets of interest;
a second channel containing a filter disposed therein; and
a third channel containing a nucleic acid binding matrix disposed therein;
wherein a first end of the cartridge is configured to directly contact a sample comprising a biological material, and
wherein a second end of the cartridge is configured to connect in a reversible fashion to a flow-through automated instrument that controls fluid flow through the cartridge.

25. The nucleic acid extraction and purification cartridge of claim 24, wherein the second and third channels are fluidically connected to the first channel.

26. The nucleic acid extraction and purification cartridge of claim 24, wherein at least one of the channels contains a dried reagent disposed therein for lysis of a sample or liberation of target bound to ligand in the first channel.

27. The nucleic acid extraction and purification cartridge of claim 26, wherein the dried reagent is an enzyme.

28. The nucleic acid extraction and purification cartridge of claim 24, wherein at least one of the channels is configured to receive vibration and/or sonication to disrupt a sample.

29. The nucleic acid extraction and purification cartridge of claim 24, wherein at least one of the channels contains particles disposed therein to aid in the disruption of a sample.

30. The nucleic acid extraction and purification cartridge of claim 24, wherein the filter comprises a porous material such as nylon, PTFE, or nitrocellulose.

31. The nucleic acid extraction and purification cartridge of claim 24, wherein the filter is designed to retain targets of interest but allows other material, including fluids, to pass through.

32. The nucleic acid extraction and purification cartridge of claim 24, wherein the nucleic acid binding matrix comprises silica, borosiliate glass, sol-gel, silanol-functionalized surface, or an anion-exchange resin.

33. The nucleic acid extraction and purification cartridge of claim 24, wherein the nucleic acid binding matrix comprises a nucleic acid binding probe or aptamer.

34. The nucleic acid extraction and purification cartridge of claim 24, wherein the cartridge is single-use.

35. The nucleic acid extraction and purification cartridge of claim 24, wherein the first channel of the cartridge selectively enriches for targets of interest by having a surface functionalized with a ligand, such as an antibody or aptamer, or contains an I-shape pillar array, or is configured for spiral inertial microfluidics, acoustofluidic bacterial separation, deterministic lateral displacement (DLD), or other microfluidic techniques for enrichment.

36. An automated flow-through instrument comprising a controller, pump, valve, flow channel, and a housing connector to reversibly connect the nucleic acid extraction and purification cartridge of any one of claims 1, 11, and 24.

37. The automated flow-through instrument according to claim 36, wherein the instrument is configured to detect a pressure change in a flow channel of the cartridge and to adjust a microfluidic protocol based on the pressure change.

38. The automated flow-through instrument according to claim 36, wherein the instrument is configured to pass heated fluids and/or air through the cartridge.

39. The automated flow-through instrument according to claim 36, wherein the instrument is configured to control vibration and/or sonication of the cartridge to promote sample disruption.

40. The automated flow-through instrument according to claim 36, wherein the instrument contains a detector to detect a target enriched or bound to the ligand in the cartridge.

41. The automated flow-through instrument according to claim 36, wherein the instrument contains a component for detection of a target nucleic acid, including qPCR, sequencing, digital PCR, and isothermal amplification.

42. The automated flow-through instrument according to claim 36, wherein the instrument reports results.

43. The automated flow-through instrument according to claim 36, wherein the instrument comprises containers to hold reagents and waste fluids.

44. The automated flow-through instrument according to claim 36, wherein the instrument is configured to use a plurality of reagents selected from lysis buffer, wash buffer, alcohol, molecular amplification and detection reagents, enzymes, elution buffer, water, oil, decontamination fluid, and air.

45. The automated flow-through instrument according to claim 36, wherein the instrument contains components to assist in the merging of two aqueous flow streams and the separation of an aqueous solution into segments that are kept separated in a flow channel using intervening air or oil.

46. The automated flow-through instrument according to claim 36, wherein the instrument contains decontamination buffers to clean re-usable components that come in contact with sample after every sample is processed.
